(19) United States
(12) Patent Application Publication
(10) Pub. No.: US 2023/0191043 A1
(43) Pub. Date: Jun. 22, 2023

(54) BLOOD VESSEL VISUALIZATION APPARATUS, BLOOD VESSEL PUNCTURE SYSTEM, AND OBSERVATION WINDOW MEMBER

(71) Applicant: TERUMO KABUSHIKI KAISHA, Tokyo (JP)

(72) Inventors: Shota YAMAMOTO, Mitaka-shi (JP); Takayuki YOKOTA, Chuo-shi (JP)

(73) Assignee: TERUMO KABUSHIKI KAISHA, Tokyo (JP)

(21) Appl. No.: 18/113,531

(22) Filed: Feb. 23, 2023

Related U.S. Application Data
(63) Continuation of application No. PCT/JP2021/030496, filed on Aug. 20, 2021.

(30) Foreign Application Priority Data
Aug. 26, 2020 (JP) 2020-142288

(51) Int. Cl. A61M 5/42

(52) U.S. Cl. CPC A61M 5/427

(57) ABSTRACT

A blood vessel visualization apparatus, a blood vessel puncture system, and an observation window member include an observation window that contains a wavelength conversion material for converting near infrared light into visible light, and that is configured to visualize an image of the near infrared light that has been radiated from a light source that irradiates with near infrared light a visualization target site at which a blood vessel of a living body is visualized, and that has then passed through the visualization target site.

### CROSS-REFERENCE TO RELATED APPLICATIONS

This application is a bypass continuation of PCT Application No. PCT/JP2021/030496, filed on Aug. 20, 2021, which claims priority to Japanese Application No. 2020-142288, filed on Aug. 26, 2020. The contents of these applications are incorporated by reference in their entireties.

### BACKGROUND

The present disclosure relates to a blood vessel visualization apparatus, a blood vessel puncture system, and an observation window member that irradiate a living body with near infrared light and visualize blood vessels.

A medical worker performs a procedure of puncturing a patient's blood vessel with a puncture needle such as an indwelling needle. It is demanded to improve a puncture success rate by making it possible to reliably insert a puncture needle into a blood vessel having an appropriate thickness when performing puncturing with the puncture needle.

In order to improve such the puncture success rate, there have been proposed various blood vessel visualization apparatuses that can irradiate a puncture target site with near infrared light having high permeability with respect to a living body, and grasp a course of a blood vessel.

For example, Japanese Patent Application Laid-Open No. 2017-64094 A discloses a blood vessel visualization apparatus employing a configuration in which light sources irradiate a patient's puncture target site with near infrared light, an image capturing means captures an image of the near infrared light reflected by the puncture target site, and a liquid crystal display apparatus visualizes and displays the captured image of the near infrared light.

### SUMMARY

However, the conventional blood vessel visualization apparatus has a problem that an apparatus configuration such as image capturing means and a liquid crystal display apparatus is necessary, and the apparatus configuration becomes complicated and large.

Therefore, a blood vessel visualization apparatus, a blood vessel puncture system, and an observation window member that can visualize blood vessels with a simpler apparatus configuration are desired.

According to one aspect of the present disclosure, a blood vessel visualization apparatus includes: a light source that irradiates with near infrared light a visualization target site at which a blood vessel of a living body is visualized; and an observation window that contains a wavelength conversion material for converting the near infrared light into visible light, an arrangement area for arranging the visualization target site is provided between the light source and the observation window, and the observation window visualizes an image of the near infrared light that has transmitted through the visualization target site arranged in the arrangement area.

According to another aspect, a blood vessel puncture system includes: the blood vessel visualization apparatus according to the above aspect; and a medical tool that can puncture a visualization target site.

According to another aspect, an observation window member of a blood vessel visualization apparatus includes a light source that irradiates with near infrared light a visualization target site at which a blood vessel of a living body is visualized, and that visualizes the blood vessel by converting the near infrared light that has transmitted through the visualization target site into visible light, and is the observation window member that includes: an observation window that contains a wavelength conversion material for converting the near infrared light into the visible light; a frame member that holds the observation window; and a support member that forms between the light source and the observation window an arrangement area for arranging the visualization target site, and the observation window visualizes an image of the near infrared light that has transmitted through the visualization target site arranged in the arrangement area.

The blood vessel visualization apparatus, the blood vessel puncture system, and the observation window member according to the above aspect can visualize blood vessels with a simple apparatus configuration.

### BRIEF DESCRIPTION OF DRAWINGS

FIG. 1 is an explanatory view of a blood vessel visualization apparatus according to a first embodiment;

FIG. 2 is a cross-sectional view taken along a line II-II of the blood vessel visualization apparatus in FIG. 1;

FIG. 3 is an explanatory view of a blood vessel puncture system including the blood vessel visualization apparatus in FIG. 1;

FIG. 4A is a cross-sectional view of a blood vessel visualization apparatus according to a second embodiment, and FIG. 4B is a perspective view of the blood vessel visualization apparatus in FIG. 4A; and

FIG. 5 is an explanatory view of a blood vessel visualization apparatus according to a third embodiment.

### DETAILED DESCRIPTION

Hereinafter, preferred embodiments of the present invention will be described in detail with reference to the accompanying drawings.

### First Embodiment

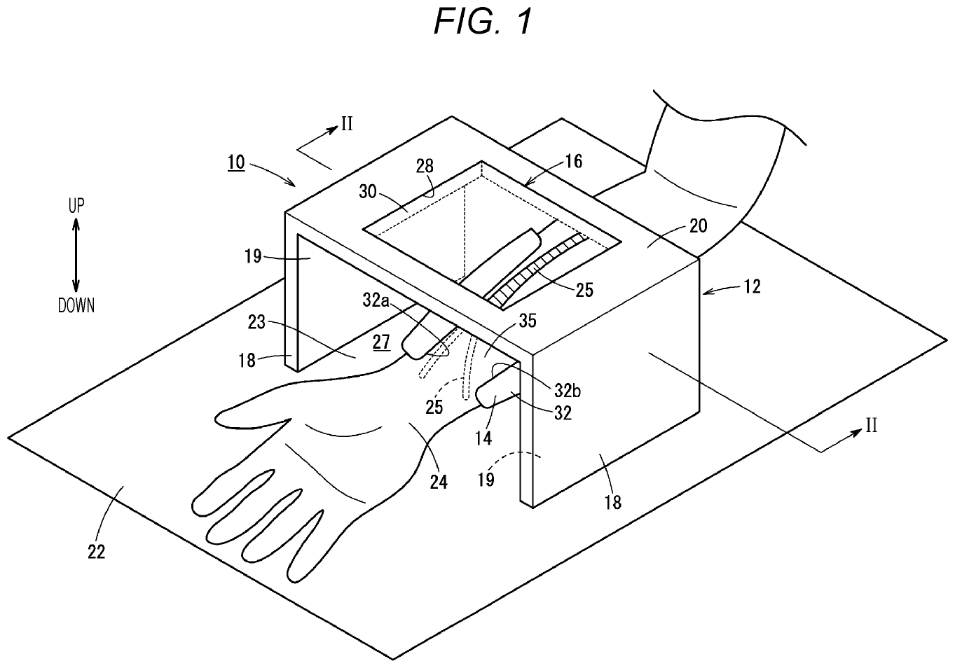

As illustrated in FIG. 1, a blood vessel visualization apparatus 10 according to the present embodiment includes a box-shaped observation window member 12, a light source 14 arranged facing an observation window member 12, and an observation window 16 provided to the observation window member 12. The observation window member 12 includes a pair of plate-like support members 18, and a plate-like frame member 20 provided so as to be bridged between the support members 18.

The support members 18 support the frame member 20 such that the frame member 20 is substantially parallel to a flat support base 22, such as a top surface of a work table, when lower end portions of the support members 18 are placed on the support base 22. The support members 18 keep a state in which the frame member 20 and the support base 22 are spaced apart from each other to form below the observation window 16 an arrangement area 23 for arranging a visualization target site 24. A separation distance between the frame member 20 and the support base 22 is determined by heights of the support members 18. Note that the separation distance between the frame member 20 and the support base 22 may be appropriately adjustable according to the size of the visualization target site 24, a specification of the light source 14, and the like.

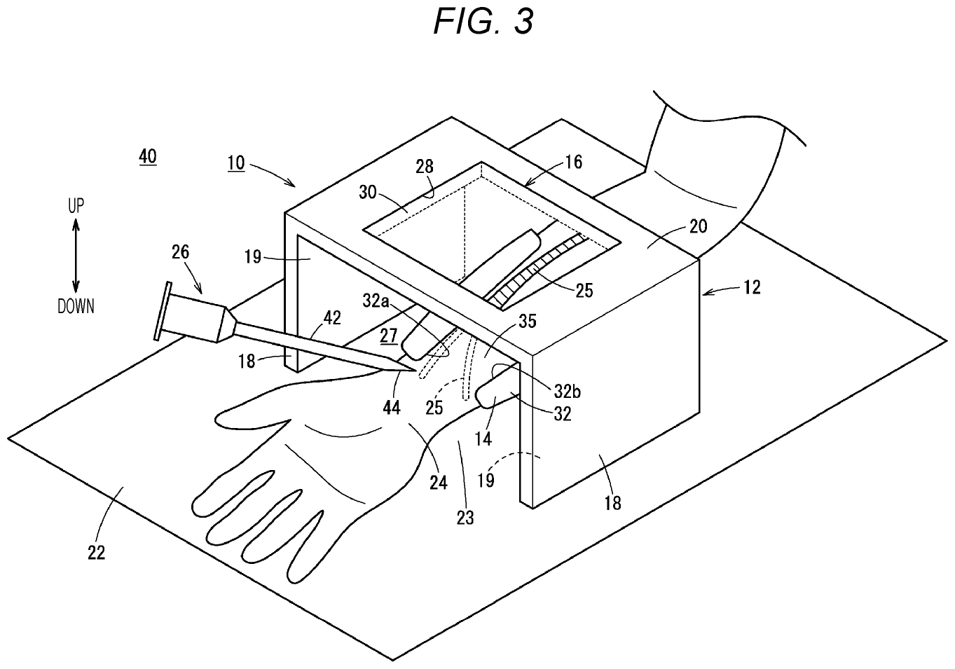

The arrangement area 23 between the frame member 20 and the support base 22 has a size in which the visualization target site 24 at which a blood vessel 25 of a living body is visualized can be arranged. The heights of the support members 18 that determine the size of the arrangement area 23 is formed to be larger than the diameter of the visualization target site 24. Thus, a puncture space 27 that enables a medical tool 26 (FIG. 3) such as a puncture needle or a catheter assembly to perform puncturing is formed between the frame member 20 and the visualization target site 24.

The visualization target site 24 is not particularly limited, yet may be a patient's arm as illustrated in FIG. 1. Furthermore, in a case in which the visualization target site 24 is an arm, the heights of the support members 18, that is, the separation distance between the support base 22 and the frame member 20 can be set to, for example, approximately 10 to 20 cm.

Inner surfaces 19 of the support members 18 on the visualization target site 24 side may be covered with a near infrared light absorbing material having a high light absorption rate of near infrared light to prevent diffused reflection of the near infrared light. Furthermore, a bottom surface 21 of the frame member 20 on the visualization target site 24 side may also be covered with the near infrared light absorbing material similarly to the inner surfaces 19 of the support members 18. The support members 18 and the frame member 20 may be made of a light shielding material that does not allow visible light to pass to limit entry of the visible light from the outside.

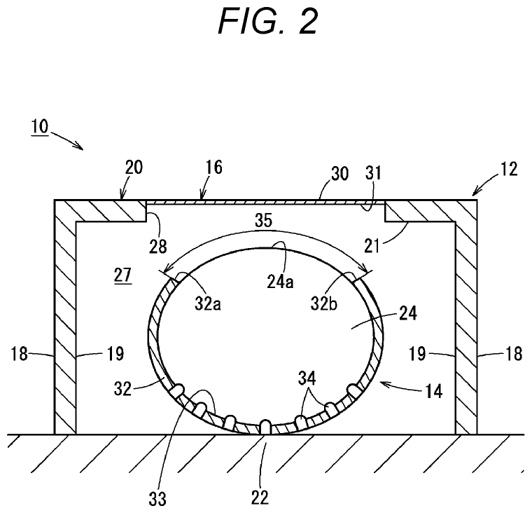

A rectangular opening portion 28 is formed in the frame member 20 of the observation window member 12. As illustrated in FIG. 2, the opening portion 28 penetrates the frame member 20 in a thickness direction. The observation window 16 is fitted and joined to the opening portion 28 of the frame member 20. The observation window 16 includes a rectangular plate member 30 formed to have dimensions that can be fitted into the opening portion 28. The thickness of the plate member 30 is not particularly limited, yet is formed equally to the thickness of the frame member 20 or thinner than the thickness of the frame member 20. The plate member 30 may be formed to be thinner than the frame member 20, and, in this case, a bottom surface 31 of the plate member 30 may be arranged at a position recessed toward an upper side of the bottom surface 21 of the frame member 20.

The surface of the plate member 30 is applied a wavelength conversion material that converts near infrared light into visible light. The wavelength conversion material that converts near infrared light into visible light is also referred to as an up-conversion material, and a material described in, for example, Japanese Patent Application Laid-Open No. 2019-172992 A can be used as the up-conversion material.

As the up-conversion material, a material containing, in molecules, a sensitizer that absorbs light of a long wavelength and efficiently generates a triplet, and an emitter that generates an excited triplet of a long life span by triplet-triplet energy transfer (TTET), further generates an excited singlet by excited triplet-triplet collision, and thereby emits fluorescence can highly efficiently convert near infrared light into visible light, and therefore is gaining attention. The observation window 16 containing the material that can highly efficiently convert near infrared light into visible light can visualize with visible luminance an image of the near infrared light that has transmitted through an inside the living body.

The observation window 16 is transparent or semitransparent with respect to visible light, and makes it possible to directly visually check the visualization target site 24 such as an arm irradiated with the visible light having entered a gap between the observation window member 12 and the visualization target site 24. When the light source 14 is turned on, a visualized image of the near infrared light is superimposed on a visible light image of light (visible light) entering from the outside to visibly project the blood vessel 25 on the observation window 16.

The observation window 16 is not limited to an observation window obtained by applying a wavelength conversion material to the surface of the plate member 30 made of a plate-like transparent material (resin or glass). The observation window 16 may be formed by, for example, dispersing a wavelength conversion material in a transparent material that forms the plate member 30. Furthermore, the observation window 16 is not limited to the plate member 30, and may be formed as a wavelength conversion film obtained by containing or applying a wavelength conversion material in or to a resin film having flexibility.

The light source 14 includes a light source housing 32, and light emitting elements 34 provided to the light source housing 32. The light source housing 32 is a plate-like member whose cross section is formed to curve in a C shape, and a slit-like gap 35 is formed between one side edge portion 32a and another side edge portion 32b. The light source housing 32 is elastically deformable, and, by deforming the side edge portion 32a and the side edge portion 32b so as to spread out, it is possible to insert the visualization target site 24 such as the patient's arm into an inner surface 33 side of the light source housing 32. The light source housing 32 winds around the visualization target site 24 by an elastic restoring force of the light source housing 32. In a state in which the light source housing 32 is attached to the visualization target site 24 of the living body, part of a surface 24a of the visualization target site 24 is exposed from a gap 35 of the light source housing 32.

One or a plurality of the light emitting elements 34 are provided on the inner surface 33 of the light source housing 32. Although not particularly limited, the plurality of light emitting elements 34 may be provided to irradiate the visualization target site 24 with uniform luminance. The light emitting elements 34 are arranged near the center of the light source housing 32 so as to face the gap 35 with the visualization target site 24 interposed therebetween. When going around the visualization target site 24 without transmitting therethrough, and entering the observation window 16, near infrared light of the light emitting elements 34 becomes noise, visibility of the blood vessel 25 lowers, and therefore the light emitting elements 34 are arranged so as to be in contact with the visualization target site 24.

The light emitting elements 34 are, for example, infrared LEDs that radiates near infrared light or near infrared laser oscillation elements, and radiate near infrared light whose center wavelength is, for example, 700 nm or more and 2500 nm or less, preferably 700 nm or more and 1400 nm or less, and more preferably 780 nm or more and 940 nm or less. The near infrared light in this wavelength band is excellent in permeability to living tissues, but is easily absorbed by red blood cells. Consequently, it is possible to visualize the blood vessel 25 containing many red blood cells as a shadow. The light source housing 32 is provided with an unillustrated power supply circuit, and this power supply circuit turns on the light emitting elements 34.

The light source 14 is arranged below the observation window 16 in a state in which the light source 14 is attached to the visualization target site 24. The light source housing 32 is arranged such that the gap 35 faces toward the observation window 16 side. At this time, the light emitting elements 34 are arranged so as to face the observation window 16 with the visualization target site 24 interposed therebetween.

The blood vessel visualization apparatus 10 according to the present embodiment is configured as described above. Next, a blood vessel puncture system 40 will be described with reference to FIG. 3.

As illustrated in FIG. 3, the blood vessel puncture system 40 includes the medical tool 26 in addition to the above blood vessel visualization apparatus 10. The medical tool 26 is a puncture needle or a catheter assembly, and includes on a distal end side thereof a puncture portion 42 that punctures the visualization target site 24. A needle tip 44 that can perform puncturing subcutaneously is formed at a distal end of the puncture portion 42. The medical tool 26 is formed in such a size that the medical tool 26 can be inserted in the puncture space 27 between the visualization target site 24 and the observation window 16, and the puncture portion 42 and the needle tip 44 can be visually checked through the observation window 16.

The blood vessel visualization apparatus 10 and the blood vessel puncture system 40 according to the present embodiment are configured as described above, and effects thereof will be described below.

As illustrated in FIG. 3, the light source 14 is first attached to the visualization target site 24 (e.g., the patient's arm) for the blood vessel visualization apparatus 10 by a user such as a doctor or a nurse. The light source 14 is attached to the visualization target site 24 by being directed such that the gap 35 of the light source housing 32 is positioned at a site that needs to be punctured with the medical tool 26. The light source 14 and the visualization target site 24 are placed on the support base 22. At this time, the visualization target site 24 is placed such that the gap 35 of the light source 14 faces upward.

Subsequently, the observation window member 12 is placed on the support base 22 so as to cover the visualization target site 24 to which the light source 14 has been attached. The support members 18 of the support base 22 are formed to have sufficiently larger dimensions than that of the visualization target site 24, and therefore the puncture space 27 that enables the medical tool 26 to perform puncturing is formed between the frame member 20 and the visualization target site 24.

Subsequently, the user performs an operation of turning on the light emitting elements 34 of the light source 14. The light emitting elements 34 radiate near infrared light. As illustrated in FIG. 2, the near infrared light emitted from the light emitting elements 34 transmits through living tissues of the visualization target site 24, and is emitted from the surface 24a of the visualization target site 24 exposed in the gap 35. When the near infrared light transmits through the living tissues of the visualization target site 24, the near infrared light is absorbed more at a portion of the blood vessel 25 containing many red blood cells, and therefore luminance of the near infrared light lowers at the portion of the blood vessel 25.

The near infrared light emitted from the surface 24a of the visualization target site 24 is converted into visible light in the observation window 16. The observation window 16 projects a visualized image in which a portion of the blood vessel 25 having low luminance becomes a relatively dark shadow, and other tissue portions are displayed relatively brightly.

Indoor visible light enters the visualization target site 24 through a gap of the observation window member 12 and the observation window 16, so that the visualization target site 24 can be visually checked through the observation window 16. The observation window 16 makes it possible to visually check a state in which the shadow of the blood vessel 25 based on the near infrared light is superimposed on a shape of the visualization target site 24 shown by the visible light.

Subsequently, the user punctures the blood vessel 25 of the visualization target site 24 with the medical tool 26 such as the puncture needle or the catheter assembly. The user can easily find the blood vessel 25 having an appropriate thickness suitable for puncturing based on the shape of the blood vessel 25 on the observation window 16. Furthermore, the user can directly visually check the visualization target site 24 and the blood vessel 25 inside the visualization target site 24 through the observation window 16 without moving a line of sight, so that it is possible to more easily and reliably puncture the blood vessel 25 with the medical tool 26.

The blood vessel visualization apparatus 10, the blood vessel puncture system 40, and the observation window member 12 according to the present embodiment provide the following effects.

The blood vessel visualization apparatus 10 according to the present embodiment includes the light source 14 that irradiates with near infrared light the visualization target site 24 at which the blood vessel 25 of the living body is visualized, and the observation window 16 that contains the wavelength conversion material for converting the near infrared light into visible light, the arrangement area 23 for arranging the visualization target site 24 is provided between the light source 14 and the observation window 16, and the observation window 16 visualizes an image of the near infrared light that has transmitted through the visualization target site 24 arranged in the arrangement area 23.

According to the above configuration, it is possible to visualize the blood vessel 25 with a simple apparatus configuration without using an image capturing apparatus, a display apparatus, or the like. In addition, since the light source 14 is arranged at the position facing the observation window 16 with the visualization target site 24 interposed therebetween, and is arranged in contact with the visualization target site 24, it is possible to reduce entry of the near infrared light that does not pass through living tissues, and visualize on the observation window 16 only the near infrared light that has transmitted through the visualization target site 24, so that it is possible to improve the visibility of the blood vessel 25.

In the above blood vessel visualization apparatus 10, the puncture space 27 that enables the medical tool 26 to puncture the visualization target site 24 may be formed. Consequently, it is preferable that it is possible to puncture the blood vessel 25 with the medical tool 26 while visually observing the blood vessel 25 through the observation window 16 without moving the line of sight.

The above blood vessel visualization apparatus 10 may further include the support members 18 that support the observation window 16 such that the observation window is spaced apart from the visualization target site 24, and the puncture space 27 may be provided between the observation window 16 and the visualization target site 24. According to this configuration, it is possible to arrange the medical tool 26 in the puncture space 27 between the observation window 16 and the visualization target site 24, and puncture the blood vessel 25 with the medical tool 26 while visually checking the medical tool 26 and the visualization target site 24 through the observation window 16.

In the above blood vessel visualization apparatus 10, the observation window 16 may be formed by applying the wavelength conversion material to the plate-like transparent material (resin or glass). According to this configuration, it is possible to visualize the near infrared light that has transmitted through the visualization target site 24 by the wavelength conversion material on the surface of the observation window 16.

In the above blood vessel visualization apparatus 10, the observation window 16 may contain the wavelength conversion material in the resin film having flexibility. According to this configuration, it is possible to make the observation window 16 thinner.

In the above blood vessel visualization apparatus 10, the observation window 16 may be transparent or semitransparent with respect to the visible light. According to this configuration, it is possible to directly visually check the visualization target site 24 with the visible light through the observation window 16, so that the user can more reliably grasp a puncture position of the medical tool 26.

The blood vessel puncture system 40 according to the present embodiment includes the above blood vessel visualization apparatus 10, and the medical tool 26 that can puncture the visualization target site 24. According to this configuration, the user can perform puncturing with the medical tool 26 while visually checking the blood vessel 25.

In the above blood vessel puncture system 40, the medical tool 26 may be the puncture needle or the catheter assembly.

The observation window member 12 according to the present embodiment is the observation window member 12 of the blood vessel visualization apparatus 10 that includes the light source 14 that irradiates with near infrared light the visualization target site 24 at which the blood vessel 25 of the living body is visualized, and that visualizes the blood vessel 25 by converting the near infrared light that has transmitted through the visualization target site 24 into visible light, and includes the observation window 16 that contains the wavelength conversion material for converting the near infrared light into the visible light, the frame member 20 that holds the observation window 16, and the support members 18 that form between the light source 14 and the observation window 16 the arrangement area 23 for arranging the visualization target site 24, and the observation window 16 visualizes an image of the near infrared light that has transmitted through the visualization target site 24 arranged in the arrangement area 23.

According to the above observation window member 12, it is possible to visualize the blood vessel 25 with a simple apparatus configuration.

### Second Embodiment

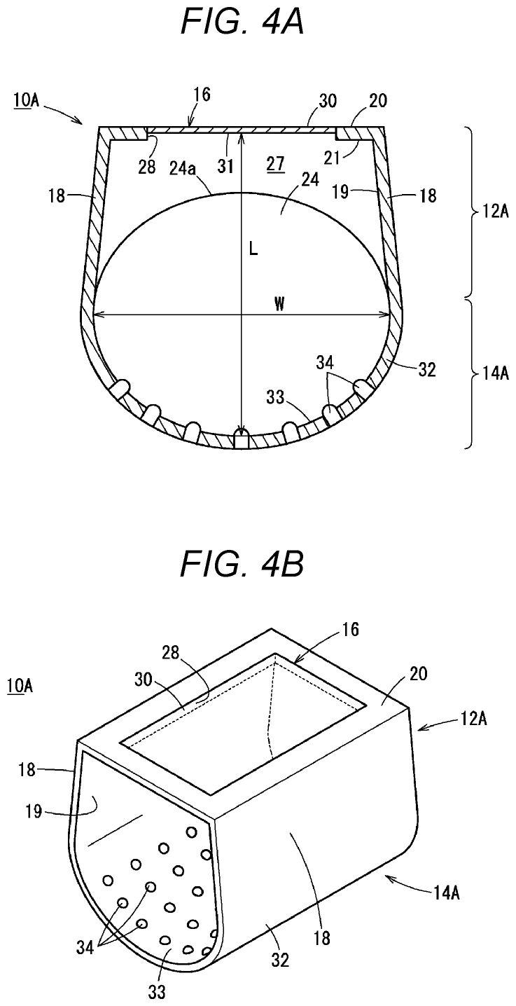

As illustrated in FIGS. 4A and 4B, in a blood vessel visualization apparatus 10A according to the present embodiment, an observation window member 12A and a light source 14A are integrated. In this regard, the same components of the blood vessel visualization apparatus 10A as those of a blood vessel visualization apparatus 10 described with reference to FIGS. 1 to 3 will be assigned the same reference numerals, and the detailed description thereof will be omitted.

As illustrated in FIGS. 4A and 4B, the observation window member 12A of the blood vessel visualization apparatus 10A includes a frame member 20 that holds an observation window 16, and a pair of support members 18 that support the frame member 20. Furthermore, the light source 14A includes a plate-shaped light source housing 32 whose cross section is formed to curve in a C shape, and a plurality of light emitting elements 34 that are provided on an inner surface 33 of the light source housing 32.

The support members 18 of the observation window member 12A and the light source housing 32 are integrally joined. A distance L between the light source housing 32 and the frame member 20 is formed to have a dimension larger than a width W of the light source housing 32 to make it possible to form a puncture space 27 between a visualization target site 24 and the observation window 16 when the visualization target site 24 is arranged.

The blood vessel visualization apparatus 10A according to the present embodiment is configured as described above, and can provide the same effects as those of the blood vessel visualization apparatus 10 described with reference to FIGS. 1 to 3.

### Third Embodiment

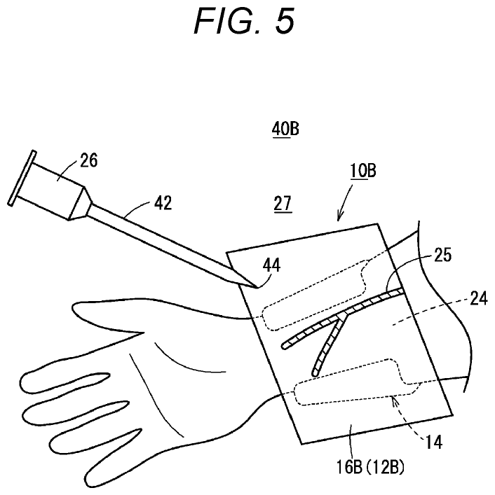

As illustrated in FIG. 5, a blood vessel visualization apparatus 10B and a blood vessel puncture system 40B according to the present embodiment include an observation window 16B arranged so as to be in contact with a visualization target site 24. An observation window member 12B includes only the observation window 16B. In this regard, the same components of the blood vessel visualization apparatus 10B as those of a blood vessel visualization apparatus 10 described with reference to FIGS. 1 to 3 will be assigned the same reference numerals, and the detailed description thereof will be omitted.

The blood vessel visualization apparatus 10B includes the observation window 16B and a light source 14. The observation window 16B is formed as a wavelength conversion film obtained by containing or applying a wavelength conversion material in or to a resin film having flexibility. The observation window 16B is transparent or semitransparent, makes it possible to visually check the visualization target site 24 by visible light, and converts near infrared light having being emitted from the light source 14 and that has transmitted through the visualization target site 24 into visible light to project.

The observation window 16B is arranged so as to be in contact with a surface 24a of the visualization target site 24. If necessary, the observation window 16B can also be arranged by being wound around the visualization target site 24. Furthermore, the observation window 16B is made of a thin resin film, and therefore can be punctured with a puncture needle. Consequently, a user can puncture a blood vessel 25 of the visualization target site 24 with a medical tool 26 through the observation window 16B. That is, according to the blood vessel visualization apparatus 10B according to the present embodiment, a puncture space 27 is formed outside (above) the visualization target site 24 and the observation window 16B (observation window member 12B).

The blood vessel visualization apparatus 10B according to the present embodiment is configured as described above, and provides following effects.

According to the blood vessel visualization apparatus 10B according to the present embodiment, the puncture space 27 is provided above the observation window 16B. According to this configuration, the wide puncture space 27 for operating the medical tool 26 can be secured, so that it is easy to operate the medical tool 26.

According to the above blood vessel visualization apparatus 10B, the observation window l6B is arranged in contact with the visualization target site 24. It is preferable that, by arranging the observation window l6B near the visualization target site 24 in this manner, the near infrared light that has transmitted through the visualization target site 24 can be converted into visible light without waste, so that it is possible to clearly project the blood vessel 25, and improve visibility of the blood vessel 25.

According to the blood vessel visualization apparatus 10B, the observation window 16B may be formed to allow the medical tool 26 to puncture therethrough, and may be configured such that the observation window 16B itself can be punctured together with the visualization target site 24 from the puncture space 27. According to this configuration, the position at which the blood vessel 25 is projected matches with a puncture position, so that the user can more accurately puncture the blood vessel 25 with the medical tool 26.

Although embodiments of the present invention have been described above with reference to preferred embodiments, the present invention is not limited to the above embodiments, and it goes without saying that various modifications can be made without departing from the gist of the present invention.

What is claimed is:

1. A blood vessel visualization apparatus comprising:
a light source configured to irradiate, with near infrared light, a visualization target site at which a blood vessel of a living body is visualized; and
an observation window that contains a wavelength conversion material for converting the near infrared light into visible light, wherein:
an arrangement area for arranging the visualization target site is located between the light source and the observation window, and
the observation window configured to visualize an image of the near infrared light that has transmitted through the visualization target site arranged in the arrangement area.

2. The blood vessel visualization apparatus according to claim 1, wherein:
the light source is configured to contact the visualization target site.

3. The blood vessel visualization apparatus according to claim 1, wherein:
a puncture space that enables a medical tool to puncture the visualization target site is formed.

4. The blood vessel visualization apparatus according to claim 3, further comprising:
a support member that supports the observation window such that the observation window is spaced apart from the visualization target site, wherein the puncture space is formed between the observation window and the visualization target site.

5. The blood vessel visualization apparatus according to claim 3, wherein:
the puncture space is provided above the observation window.

6. The blood vessel visualization apparatus according to claim 5, wherein:
the observation window is configured to be arranged in contact with the visualization target site.

7. The blood vessel visualization apparatus according to claim 5, wherein:
the observation window is configured to allow the medical tool to puncture therethrough, and the observation window itself can be punctured together with the visualization target site from the puncture space.

8. The blood vessel visualization apparatus according to claim 1, wherein:
the observation window comprises a wavelength conversion material disposed on a plate-like transparent material.

9. The blood vessel visualization apparatus according to claim 1, wherein:
the observation window contains the wavelength conversion material in a flexible resin film.

10. The blood vessel visualization apparatus according to claim 1, wherein:
the observation window is transparent or semitransparent with respect to the visible light.

11. A blood vessel puncture system comprising:
the blood vessel visualization apparatus according to claim 1; and
a medical tool configured to puncture the visualization target site.

12. The blood vessel puncture system according to claim 11, wherein:
the medical tool is a puncture needle or a catheter assembly.

13. An observation window member of a blood vessel visualization apparatus that comprises a light source configured to irradiate with near infrared light a visualization target site at which a blood vessel of a living body is visualized, and that is configured to visualize the blood vessel by converting the near infrared light that has transmitted through the visualization target site into visible light, the observation window member comprising:
an observation window that contains a wavelength conversion material for converting the near infrared light into the visible light;
a frame member that holds the observation window; and
a support member that forms, between the light source and the observation window, an arrangement area for arranging the visualization target site,
wherein the observation window is configured to visualize an image of the near infrared light that has transmitted through the visualization target site arranged in the arrangement area.

14. A method of visualizing a blood vessel, the method comprising:
providing a blood vessel visualization apparatus comprising:
a light source configured to irradiate, with near infrared light, a visualization target site at which a blood vessel of a living body is visualized; and
an observation window that contains a wavelength conversion material for converting the near infrared light into visible light, wherein:
placing the blood vessel visualization apparatus such that an arrangement area for arranging the visualization target site is located between the light source and the observation window;
arranging the visualization target site in the arrangement area; and
visualizing, with the observation window, an image of the near infrared light that has transmitted through the visualization target site arranged in the arrangement area; and
puncturing the blood vessel with a puncture portion of a medical tool.
